(19) United States
(12) Patent Application Publication
(10) Pub. No.: US 2026/0199450 A1
(43) Pub. Date: Jul. 16, 2026

(54) Nucleic Acid-Lipid Nanoparticle Suitable for Intramuscular Administration, Preparation Thereof, and Use Thereof

(71) Applicant: CHENGDU NUOEN GENOMICS, LTD, Chengdu City, Sichuan Province (CN)

(72) Inventors: Kai XU, Houston, TX (US); Dongsheng REN, Lhasa (CN); Yucheng ZHOU, Lhasa (CN); Xiujin WU, Chengdu (CN); Yingxue LEI, Chengdu (CN); Yan ZHANG, Lhasa (CN); Liang CHEN, Chengdu (CN); Chen HE, Lhasa (CN); Yaoyi ZHANG, Chengdu (CN); Jian YANG, Chengdu (CN)

(21) Appl. No.: 19/131,914

(22) PCT Filed: Jan. 3, 2023
(86) PCT No.: PCT/CN2023/070153

§ 371 Date: May 21, 2025

(30) Foreign Application Priority Data
Nov. 21, 2022 (CN) 202211452115.5

(51) Int. Cl. A61K 39/215; A61K 9/00; A61K 9/1272; A61K 9/51; A61K 31/7105; A61K 39/00; A61K 39/145; A61P 31/14; C12N 7/00; G01N 21/64

(52) U.S. Cl. CPC A61K 39/215; A61K 9/0019; A61K 9/1272; A61K 9/5123; A61K 31/7105; A61K 39/0011; A61K 39/145; A61P 31/14; C12N 7/00; G01N 21/6486; A61K 2039/53; A61K 2039/54; A61K 2039/55555; A61K 2039/575; C12N 2760/16034; C12N 2770/20034

(57) ABSTRACT

Provided are a nucleic acid-lipid nanoparticle suitable for intramuscular administration, a preparation thereof, and use thereof. The nucleic acid-lipid nanoparticle consists of the following components: (a) at least one nucleic acid; (b) at least one ionizable lipid, accounting for 20 mol % to 35 mol % of total lipids; (c) at least one non-ionizable cationic lipid, accounting for 15 mol % to 30 mol % of the total lipids; (d) a lipid mixture of neutral phospholipids or derivatives thereof, accounting for 0 mol % to 10 mol % of the total lipids; (e) a mixture of cholesterol or derivatives thereof, accounting for 40 mol % to 56 mol % of the total lipids; and (f) a mixture of PEG or derivatives thereof, accounting for 1.5 mol % to 3 mol % of the total lipids. The nucleic acid-lipid nanoparticle can wrap mRNA or plasmid DNA and is suitable for intramuscular administration.

### FIELD OF INVENTION

The present invention relates to the field of biomedical technology, and in particular to a nucleic acid-lipid nanoparticle suitable for intramuscular administration, and its preparation and application.

### DESCRIPTION OF RELATED ARTS

The mRNA vaccines based on lipid nanoparticle (Lipid Nanoparticle, LNP) technology can stimulate both humoral immunity and cellular immunity at the same time, and are more protective and cost-effective than other types of vaccines, as well as having a larger production scale. The COVID-19 mRNA vaccines have been used by billions of people, enabling large-scale application of prophylactic mRNA vaccines in the real world. At the same time, it was also revealed that mRNA vaccination caused side effects such as fever, allergies, inflammatory storms, and liver toxicity, and its incidence was higher than that of traditional vaccines. Although all adverse reactions are mild and transient, this experience brings uncertainty to the promotion of nucleic acid vaccine technology in broad application scenarios.

Composition and active ingredients of current mRNA vaccines:

The first batch of mRNA vaccine lipid formulas that have obtained market access are all composed of an ionizable cationic lipid, a neutral phospholipid (DSPC), cholesterol, and PEG or its derivative lipids. The molar ratio of the four complies with the ratio range covered by U.S. Pat. No. 8,058,069, that is, cationic lipids, neutral phospholipids, cholesterol or its derivatives, PEG or its derivative lipids account for 50-65 mol %, 4-10 mol %, 30-40 mol %, and 0.5-2 mol % of the total lipids, respectively. Another example is the recently applied US202210336875.3 by CanSino Biologics in China, a new COVID-19 mRNA vaccine and its preparation method and use. The disclosed composition of lipid components is: cationic lipid: neutral phospholipid: steroid lipid: polyethylene glycol-lipid with a molar ratio of 30-60:5-20:20-50:0.1-10.

Activation of both humoral and cellular immunity by mRNA vaccine:

As mentioned above, compared with traditional vaccines, the COVID-19 mRNA vaccines have an extraordinary protective power, mainly because it can activate both humoral immunity and cellular immunity at the same time. Cellular immunity produces long-lasting memory cells and T-cell immunity, which is not only beneficial for preventive vaccines to work but is also the main mechanism of action that tumor vaccines rely on.

Recipients of COVID-19 mRNA vaccines (for example, mRNA-1273 vaccine) who are antibody-negative but neutralizing antibody-positive are also protected by the vaccine. According to Gilbert, P. B. et al., it is estimated that neutralizing antibodies contribute about two-thirds of the efficacy of mRNA vaccines. 58 days after vaccination, the neutralizing antibody titers NT50 are 10, 100 and 1000, which correspond to vaccination protection values of 78%, 91% and 96%. In clinical phase II, vaccine neutralizing antibody titer has been used as a form of “immune bridging” as the basis for vaccine approval and has been adopted by many countries and regions.

Systemic expression of LNP-delivered cargos as one of the sources of acute side effects of nucleic acid vaccines:

According to Patone, M. et al., Hassett, K. J. et al., and Pardi, N. et al., after intravenous administration of the mRNA-LNP preparation, the nucleic acid lipid nanoparticles mainly attack the liver tissue, and also transfection in major organs and tissues such as the lungs, brain tissue, heart, and blood vessel extremities, possessing the risk of fever, chills and other adverse effects. Before using Onpattro (MC3-LNP, Alnylam), acetaminophen should be taken in advance to deal with potential systemic inflammation and neurotoxic side effects.

On the other hand, according to Ndeupen, S. et al., nasal inhalation of LNP also led to a significant increase in toxicity. The same dose of LNP delivered by nasal inhalation can cause similar inflammatory reactions in the lungs and lead to high mortality.

Intramuscular administration offers relatively large injection area for drug administration and may therefore cause fewer adverse reactions at injection sites, making it one of the best ways to vaccine administration. Administration by intramuscular injection and subcutaneous injection showed more sustained protein expression in tissues than that by intravenous administration. Despite this, studies have confirmed that after intramuscular administration, a considerable amount of nucleic acid lipid nanoparticles are still delivered systemically through the circulatory system, targeting liver tissue, lung tissue, brain tissue, and myocardial tissue, and stimulating the production of a large amount of exogenous protein in a short period of time. The systemically delivered lipid nanoparticles and the large amount of off-target expressed exogenous proteins stimulate tissue damage, neurotoxicity, and activation of cytokines and complement, producing transient toxic side effects.

Ionizable lipids are inherently potent immune adjuvants-triggering systemic side effects:

Lipid nanoparticles themselves possess adjuvant activity. The researchers, by using mRNA and protein subunit vaccines of influenza virus and SARS-COV-2, demonstrated that empty lipid nanoparticles (without bioactive substances such as nucleic acids) display inherent adjuvant activity, which can promote the induction of strong follicular helper T cells, germinal center B cells, long-lived plasma cells, and memory B cell in mice, and are associated with the generation of long-lasting protective antibodies. Lipid nanoparticles stimulate extremely strong humoral immunity, produce excessive antibodies in a short period of time, and increase the burden on the body's immune system.

Cheng et al. added a fifth charged lipid to the LNP system to adjust the internal charge of lipid nanoparticles. In maintaining the original ratio of the four components (5A2-SC8:DOPE:Chol:PEG=15:15:30:3), DOTAP was added from 0 to 100% in proportion to make a series of LNPs varying in DOTAP contents. By intravenous injection of lipid granules, the delivered genes exhibit organ selectivity. Maintaining a certain amount of ionizable lipids in LNP can ensure sufficient adjuvant effect, especially when administered by intramuscular injection, the vaccine is ensured to have sufficient adjuvant to produce minimal immunogenicity. The above disclosure of Cheng et al. utilizes Dotap to change the surface charge of LNP, thereby changing the delivery targeting. Because it is administered by intravenous injection, in order to reduce the inflammatory response and systemic toxicity caused by ionizable lipids, its dosage has to be reduced, thereby reducing the adjuvant effect of LNP.

Ionizable lipids are the key contributing factor for the adjuvant activity of LNP, demonstrating significant dose-dependent effects. In contrast, non-ionizable cationic lipids lack adjuvant effects and fail to induce sufficient antibody titers. Notably, all charge-mediated targeting strategies mentioned above for lipid nanoparticles use intravenous administration. In order to avoid the toxicity caused by intravenous administration, the concentration of ionizable lipids has to be reduced, thereby diminishing the immunoadjuvant efficacy of LNPs, and rendering them unsuitable as the carriers for nucleic acid vaccines.

In addition, the existing LNP formulations face challenges of poor stability. mRNA-LNP products require low-temperature storage at −20° C. to −40° C., complicating vaccine distribution. To address this issue, mRNA-LNP lyophilized powder formulations are currently under development, but this method increases the complexity and cost of vaccine production. Additionally, patented methods have reported the use of synthetic thermostable imidazole-modified ionizable cationic LNPs to resolve the ultra-low temperature storage and cold chain transportation challenges of current mRNA vaccines. However, this approach also elevates manufacturing difficulties and costs.

### SUMMARY OF THE PRESENT INVENTION

One objective of the present invention is to provide a nucleic acid-lipid nanoparticle suitable for intramuscular administration to address the aforementioned issues.

To achieve the above objective, the technical solution adopted by the present invention is as follows: a nucleic acid-lipid nanoparticle suitable for intramuscular administration, comprises the following components: (a) at least one nucleic acid; (b) at least one ionizable lipid, constituting 20 mol % to 35 mol % of total lipids; (c) at least one non-ionizable cationic lipid, constituting 15 mol % to 30 mol % of the total lipids; (d) neutral phospholipids or lip mixtures of their derivatives, constituting 0 mol % to 10 mol % of the total lipids; (e) cholesterol or a mixture of its derivatives, constituting 40 mol % to 56 mol % of the total lipids; (f) PEG or a mixture of its derivatives, constituting 1.5 mol % to 3 mol % of the total lipids; molecules of (a) the nucleic acid is encapsulated within the lipid nanoparticle composed of (b), (c), (d), (e) and (f). The lipid layer of the present invention protects nucleic acids from enzymatic degradation in vivo.

The inventors of this application have experimentally demonstrated that: Incorporating an appropriate amount of non-ionizable cationic lipids into legacy LNPs can alter the delivery targeting specificity of lipid nanoparticles, reduce their systemic delivery capacity, and enhance their stability. A key feature is that, when administered via intramuscular injection, the formulation enables prolonged expression at the injection site, with the target gene primarily expressed locally, production of neutralizing antibodies and antigen-specific binding antibodies, while significantly reducing transfection and gene expression in organs such as the liver, lungs, brain, and spleen.

As mentioned above, although current studies have reported that modifying the surface charge of LNPs can alter their tissue targeting specificity, these approaches are limited to intravenous administration and result in significantly reduced adjuvant activity, rendering them unsuitable for nucleic acid vaccines. However, the present invention supplements an appropriate amount of non-ionizable cationic lipids instead of replacing ionizable lipids. Consequently, the adjuvant activity of the LNP^{⊕} formulation of the present invention provided by the ionizable lipids is preserved, making it suitable for intramuscular administration.

Additionally, the present invention demonstrates that the incorporation of an appropriate amount of non-ionizable cationic lipids enhances the stability of lipid nanoparticles. One key manifestation of this improvement is an increased tolerance to non-ionic surfactants, requiring Triton X-100 at concentrations of 10 vol % or higher to achieve complete dissociation.

As a preferred technical embodiment, the formulation comprises the following components: (a) mRNA; (b) one ionizable lipid, constituting 23.01 mol % to 24.17 mol % of the total lipids; (c) at least one non-ionizable cationic lipid, constituting 23.01 mol % to 24.17 mol % of the total lipids; (d) neutral phospholipids, constituting 4.91 mol % to 9.35 mol % of the total lipids; (e) cholesterol, constituting 42.43 mol % to 44.56 mol % of the total lipids; (f) PEG, constituting 2.20 mol % of the total lipids. In this present application, these formulations are designated as “LNP^{⊕}45” preparation (when the content of neutral phospholipid is 4.91 mol %) and “LNP^{⊕}46” preparation (when the content of neutral phospholipid is 9.35 mol %).

As a preferred technical embodiment, the formulation comprises the following components: (a) at least one nucleic acid; (b) at least one ionizable lipid, constituting 20 mol % to 35 mol % of total lipids; (c) at least one non-ionizable cationic lipid, constituting 15 mol % to 30 mol % of the total lipids; (d) cholesterol or a mixture of its derivatives, constituting 40 mol % to 56 mol % of the total lipids; (e) a PEG or a mixture of its derivatives, constituting 1.5 mol % to 3 mol % of the total lipids; the molecules of (a) the nucleic acid is encapsulated within the lipid nanoparticle composed of (b), (c), (d) and (e).

As a further preferred technical embodiment, the formulation comprises the following components: (a) a mRNA or DNA; (b) an ionizable lipid, constituting 25.45 of the total lipids; (c) a non-ionizable cationic lipid, constituting 25.45 mol % of the total lipids; (d) cholesterol, constituting 46.90 mol % of the total lipids; (e) PEG, constituting 2.20 mol % of the total lipids. In this embodiment, the formulation does not contain neutral phospholipids; and it can deliver DNA in addition to mRNA. This formulation is designated as “LNP^{⊕}25” preparation in the present application.

In another preferred embodiment, the nucleic acid-lipid nanoparticle comprises: (a) a mRNA or DNA; (b) an ionizable lipid, constituting 30.88 mol % of the total lipids; (c) a non-ionizable cationic lipid, constituting 15.35 mol % of the total lipids; (d) cholesterol, constituting 42.42 mol % of the total lipids; (e) a neutral phospholipid, constituting 4.8 mol % to 9.4 mol % of the total lipids; (f) a PEG lipid, constituting 2.20 mol % of the total lipids. In this preferred embodiment, there is no neutral phospholipids. It can deliver DNA in addition to mRNA. This formulation is designated as the “LNP^{⊕}74” preparation in the present application.

As a further preferred technical embodiment, the ionizable lipid and the non-ionizable cationic lipid are present in equimolar concentrations.

As a further preferred technical embodiment, the nucleic acid comprises at least one mRNA encoding a polypeptide or an mRNA containing modified nucleotides.

As a further preferred technical embodiment, the nucleic acid comprises DNA.

As a further preferred technical embodiment, the non-ionizable cationic lipid is selected from at least one of DOTAP, DOTMA, DC-chol and DOSPA or derivatives thereof.

As a further preferred technical embodiment, a molar ratio of the non-ionizable cationic lipid to the cholesterol ranges from 10:9 to 10:11.

The experimental results have confirmed that: when the molar ratio of cationic lipid to cholesterol in LNP formulation is 10:9 to 10:11, the formulation achieves optimal gene delivery and transfection efficiency in mice.

The present invention demonstrates that: the neutral phospholipid component in LNP exerts entirely distinct effects on the expression of mRNA and DNA. Specifically, the neutral phospholipid component enhances the expression efficacy of mRNA-LNP^{⊕} formulations, whereas the neutral phospholipid component suppresses the expression efficacy of DNA-LNP^{⊕} formulations. Consequently, the present invention avoids the inclusion of neutral phospholipid when applied to DNA delivery.

At the same time, non-ionized cationic lipids such as DOTAP can significantly enhance the expression level of the tracer gene in the DNA-LNP^{⊕} formulation at the intramuscular injection site of mice.

The second object of the present invention is to provide a formulation prepared from the aforementioned nucleic acid-lipid nanoparticle, and the technical solution adopted involves: the preparation comprises the nucleic acid-lipid nanoparticle and a pharmaceutically acceptable carrier.

As a preferred technical embodiment, the formulation is an injection. The nucleic acid-lipid nanoparticle injectable formulation of the present invention demonstrates a clear dose-dependent relationship between the ionizable lipid component in the LNPs and the delivery and sustained expression of the tracer gene at the intramuscular injection site under intramuscular administration. The intensity and duration of tracer gene expression increase with higher doses of ionizable lipids. The non-ionizable cationic lipids exhibit far inferior capability to deliver tracer genes at the injection site compared to ionizable lipids at equivalent doses and lack the ability to sustain long-term expression of tracer genes. Thus, maintaining sufficient ionizable lipid concentration and dose is a prerequisite for LNP vaccine formulations administered intramuscularly to stimulate high-titer specific antibody production.

The present invention demonstrates that: under intramuscular administration, the incorporation of non-ionizable cationic lipids such as DOTAP into lipid particles composed of various ionizable lipids, including ALC-0315, MC3, DHA-1, L319, and SM-102, can effectively balance the adjuvant activity of ionizable lipids and reduce off-target expression of nucleic acid-lipid particles in visceral tissues of mice. Combined with the similar effects on the expression pattern of LNP formulations incorporating another non-ionized cationic lipid DOTMA, by extension, these findings validate the universal applicability of non-ionizable cationic lipids in modulating off-target expression of LNP formulations while maintaining sustained expression at the intramuscular injection site. This capability extends to diverse LNP types and combinations with other non-ionizable cationic lipid molecules, such as DC-Chol, DOSPA or derivatives thereof. The controllability and predictability of this approach establish it as a modular and universal strategy for achieving effective intramuscular drug delivery.

The third object of the present invention is to provide the use of the above-mentioned nucleic acid-lipid nanoparticles in the preparation of biological vaccines

As a preferred technical solution, the biological vaccine is a COVID-19 vaccine, an influenza vaccine, or a tumor vaccine.

Specifically, the present invention has been verified through experiments that, within the range defined by the above-mentioned lipid particle preparation, the lipid molar ratio can be adjusted to change the expression ratio of the exogenous gene at the intramuscular injection site and in the visceral tissue under intramuscular administration, thereby adjusting the proportion of humoral immunity and cellular immunity produced by the preparation to adapt to different needs and increase the effective utilization of the patient's immune system. For example, therapeutic tumor vaccine preparations need to activate cellular immune responses rather than humoral immune responses. For preventive vaccines such as COVID-19 vaccine and the flu vaccine, rely on neutralizing antibody titer as the main indicator for reducing severe cases. However, since cellular immunity takes a longer time to take effect, it is necessary to balance humoral immunity and cellular immunity, appropriately enhancing humoral immunity to produce specific IgG antibodies to cope with acute pathogen infections.

The present invention has confirmed through intramuscular injection of luciferase gene transfection experiments that, compared to the corresponding LNP formulation, LNP^{⊕} formulation reduces the transfection and expression of tracer genes in visceral tissues, especially in liver tissue and brain tissue. Blood liver biochemical indicators can objectively, in real time, and accurately measure liver health status. Through intramuscular injection of COVID-19 virus S protein mRNA-LNP, the present invention detects liver damage-related indicators such as ALT, AST, and TBIL in the blood. The results shows that the traditional mRNA-LNP causes significant liver damage to mice within 48 hours after intramuscular injection. In contrast, no significant changes in blood liver damage indicators are detected in the LNP^{⊕} formulation group mice. IVIS imaging results reveal that in addition to targeting liver cells for transfection, the LNP formulations also transfect myocardium, brain tissue, and peripheral limbs to varying degrees, suggesting that the LNP formulations may also cause varying degrees of tissue damage to visceral tissues, which could be a key reason that cannot be ignored for the various side effects of mRNA vaccines. With intramuscular administration, the LNP^{⊕} formulation significantly reduced gene transfection levels in liver tissue, thus avoiding damage to liver tissue. At the same time, the LNP^{⊕} formulation also significantly reduces the gene transfection levels in other tissues such as brain tissue, respiratory tract, and peripheral limbs, suggesting reduced damage to other visceral tissues by the LNP^{⊕} formulation, and making it possible to prepare safer mRNA-LNP formulations.

The present invention provides a formulation for treating cancer, preventing cancer, or delaying the onset or progression of cancer, or alleviating symptoms associated with cancer, by administering to an individual the composition discussed in the above aspects and embodiments. In certain embodiments, the polypeptide may encode a therapeutic enhancing factor, such as an immunomodulatory molecule or other factors as previously described.

The present invention also provides a method for measuring the stability of the lipid nanoparticles described herein. Specifically, the nucleic acid lipid nanoparticle of the present invention has increased tolerance to surface detergents, requiring a Triton X-100 solution with a concentration of 10 vol % or higher to completely dissociate and separate the lipid nanoparticle from the nucleic acid they encapsulate therein. More specifically, these nucleic acid lipid nanoparticles of the present invention remain substantially intact in Triton X-100 solutions with a concentration less than 2 vol %; and completely dissociated in solutions with Triton X-100 concentrations of 10% or higher.

The present invention also provides a method for in vivo delivery of the formulation, which comprises administering the lipid nanoparticles described herein, such as nucleic acid lipid particle vaccine, to mammals and subjects via intramuscular injection and subcutaneous injection.

Compared with the prior arts, the advantages of the present invention are: the nucleic acid-lipid nanoparticles of the present invention, whose lipids can be used as nucleic acid LNP delivery carriers to encapsulate mRNA or plasmid DNA, are particularly suitable for intramuscular administration, enabling long time expression at the injection site, production of high-titer specific neutralizing antibodies, and reducing off-target expression in visceral tissues such as in the liver and spleen Additionally, they can significantly improve the temperature stability of nucleic acid-lipid nanoparticles, facilitating the transportation and distribution of mRNA vaccines, and etc.

### BRIEF DESCRIPTION OF THE DRAWINGS

FIG. 1 shows the effect of ionizable lipid MC3 and non-ionizable cationic lipid DOTAP on the expression of tracer genes at the intramuscular injection site.

FIG. 2 shows the expression pattern of tracer genes after intramuscular injection of LNP lipid particles composed of ionizable lipid ALC-0315, to which non-ionizable cationic lipid DOTAP is added.

FIG. 3 shows the enhanced expression level and duration of tracer genes at the intramuscular injection site of LNP lipid particle, to which non-ionizable cationic lipid DOTAP is added.

FIG. 4 shows the expression pattern of tracer genes after intramuscular injection of LNP lipid particle composed of ionizable lipid ALC-0315, to which non-ionizable cationic lipid DOTMA is added.

FIG. 5-FIG. 8 illustrate the expression pattern of tracer genes after intramuscular injection of LNP lipid particle composed of ionizable lipids MC3, DHA-1, L319, and SM-102, after each of which is added with non-ionizable cationic lipid DOTAP, respectively.

FIG. 9 illustrates the effect of neutral phospholipid concentration in mRNA-LNP formulations on tracer gene expression after intramuscular administration.

FIG. 10 illustrates the comparison of expression levels of LNP^{⊕} formulations at the injection site and in the abdominal cavity after intramuscular administration.

FIG. 11 illustrates the effect of neutral phospholipid concentration in DNA-LNP formulation on tracer gene expression after intramuscular administration.

FIG. 12 illustrates the effect of cholesterol concentration in mRNA-LNP formulation on tracer gene expression after intramuscular administration.

FIG. 13 illustrates the effect of PEG concentration in mRNA-LNP formulation on tracer gene expression after intramuscular administration.

FIG. 14 shows the ELISA results for S protein-specific IgG antibody in mouse serum collected at 21 days (3wp1) after the first immunization and 7 days (1wp2), 14 days (2wp2), and 21 days (3wp2) after the second immunization.

FIG. 15 shows the ELISA results for RBD-ACE2 competitive neutralizing antibody in mouse serum collected at 21 days (3wp1) after the first immunization and 7 days (1wp2), 14 days (2wp2), and 21 days (3wp2) after the second immunization.

FIG. 16 shows the liver-related serological indicators of Balb/C mice under intramuscular administration of nCovS2P@LNP formulation.

FIG. 17 illustrates the structure of LNP^{⊕}46 lipid particle of the present invention.

### DETAILED DESCRIPTION OF THE PREFERRED EMBODIMENT

Before describing the present invention, the following definitions are provided to help understand the present invention. Unless otherwise defined, all technical and scientific terms used herein have the same meaning as commonly understood by those of ordinary skilled in the field to which the present invention belongs.

Ionizable lipid: Also known as ionizable cationic lipid, which is amphiphilic molecule with hydrophilic group and hydrophobic group, consisting of a polar head (hydrophilic group), a linker bond, and a hydrophobic tail. The hydrophilic head of an ionizable lipid is composed of a tertiary amine, which exhibits degrees of protonation at different pH values, rendering it ionizable. Ionizable lipid used in the present invention include but are not limited to: ALC-0315, Dlin-MC3-DMA (MC3), Lipid L319, SM-102, DHA-1, and the like.

Non-ionizable cationic lipid: An amphiphilic molecule with hydrophilic and hydrophobic groups, consisting of a polar head (hydrophilic group), a linker bond, and a hydrophobic tail. The hydrophilic head is a quaternary ammonium salt, functioning as a permanent cation without ionizable properties. The non-ionizable lipids used in the present invention include but are not limited to: DOTAP ((2,3-dioleoyl-propyl)-trimethylammonium-chloride); DOTMA (trimethyl-2,3-dioleyloxypropylammonium chloride); DC-Chol (3β-[N—(N,N-dimethylaminoethyl) carbamoyl]cholesterol); DOSPA; or one of their derivatives.

Neutral phospholipid: An amphiphilic phosphatidylcholine with a hydrophilic head and a hydrophobic tail. Commonly used synthetically modified phospholipids include: DSPC, DOPE, DOPC, ePC, their derivatives, and the like.

Cholesterol: A natural small-molecule lipid, a primary component of cell membranes.

LNP: Lipid Nanoparticle. Lipid nanoparticles are composed of at least one ionizable lipid and at least one neutral phospholipid, encapsulating biologically active molecules such as nucleic acids. Biologically active molecules may include RNA, DNA, siRNA, miRNA, proteins, peptides, and the like.

LNP^{⊕}: LNP containing at least one non-ionizable cationic lipid and at least one ionizable lipid. The bioactive molecules encapsulated and delivered by the LNP^{⊕}s are identical to those in the LNP formulations described above.

Nucleic acid: A polymer containing at least two deoxyribonucleotides or ribonucleotides in single-stranded or double-stranded form, including DNA and RNA. RNA may take forms of siRNA, microRNA (miRNA), mRNA, tRNA, rRNA, tRNA, circular RNA or combinations thereof. Nucleic acid can be synthetic, naturally occurring or non-naturally occurring. Examples of such analogs include, but are not limited to, phosphorothioate, phosphoramidate and peptide nucleic acid (PNA), and they may contain known natural nucleotide analogs as well as artificially modified nucleotides, such as pseudouridine, methylated nucleotide, or methylpseudouridine-modified nucleotides. DNA may include double-stranded DNA, single-stranded DNA, plasmid DNA, and the like.

nCovS: SARS-COV-2 Spike protein gene.

nCovS2P: A recombinant gene encoding the pre-fusion conformation-locked SARS-Cov-2 Spike protein, modified with point mutations K986P and V987P.

mRNA vaccine: mRNA-LNP formulation based on LNP formula encapsulating mRNA, generally administered by intramuscular injection to produce specific antigens in the subject's body, inducing the production of specific antibodies, thereby generating immune protection.

Fluc: Firefly luciferase gene, the gene encoding firefly luciferase.

IV: Intravenous injection administration, specifically via tail vein injection in mice in the present invention.

IM: Intramuscular injection administration, specifically into the hind limb muscle tissue of mice in the present invention.

mRNA: A eukaryotic messenger RNA, a single-stranded RNA composed of a 5′-m7G cap, 5′-UTR, translation start codon, coding region, stop codon, 3′-UTR, and polyadenylate tail, serving as a template for protein translation.

BNT162b2: A recombinant mRNA sequence of the Covid-19 Spike protein used in the Pfizer/BioNTech mRNA vaccine, modified with the S2P mutation.

IVT: In vitro transcription (In vitro transcription).

The present invention is further described below in conjunction with specific embodiments. It should be understood that these embodiments are only used to illustrate and not to limit the present invention.

### Embodiment 1

### Raw Materials and Preparation of Formulation

### 1.1: Preparation of RNA

According to this Embodiment of the present invention, the mRNA used is obtained through IVT reactions. The general process involves enzymatic digestion of plasmid DNA template followed by column purification to obtain linearized plasmid DNA. RNZ is produced via IVT transcription (Thermo Fisher, MEGAscript® Kit). After transcription, RNA is purified using a Monarch® RNA Cleanup Kit. Unless otherwise specified, the transcription reaction substrate UTP is replaced by N1-methylpseudouridine (m1ψ).

The capping modification of mRNA is performed using the capping enzyme from Vaccinia virus from NovoProtein (Vaccinia Capping Enzyme). The mRNA capping reaction is set up according to the kit's recommended system, and incubated at 37° C. for 1 hour. After the reaction is completed, the capped product is purified using a Monarch® RNA Cleanup Kit. The purified mRNA is dissolved in sterile water for injection, analyzed by RNA gel electrophoresis and evaluated by Qubit concentration measurement.

### 1.2: Preparation of Plasmid DNA

The plasmid DNA used in this Embodiment of the present invention is obtained using the Qiagen EndoFree Plasmid Maxi Kit.

### 1.3: Preparation of LNP and LNP^{⊕} Formulations

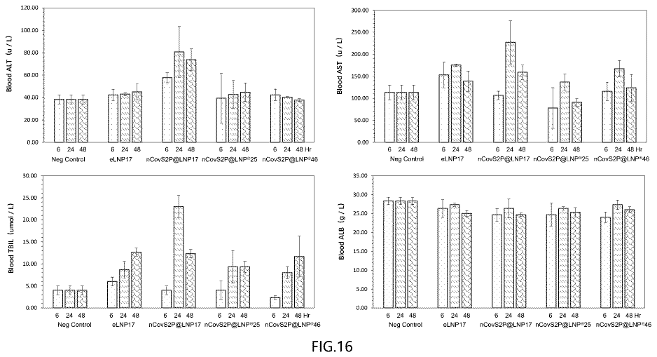

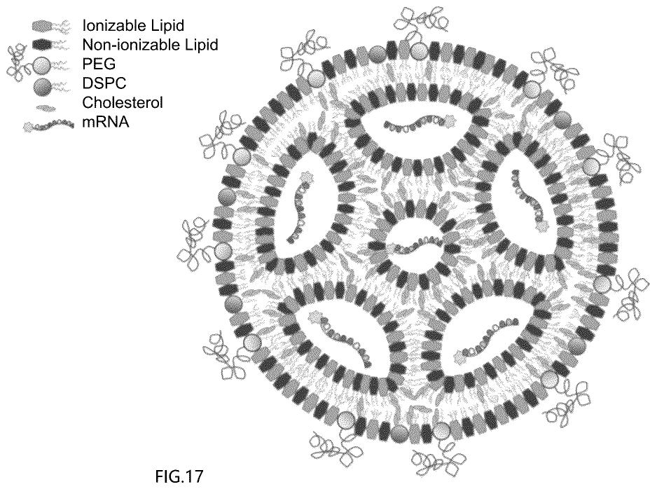

The LNP and LNP^{⊕} formulations consist of ionizable lipids, non-ionized cationic lipids, DSPC, cholesterol and PEG2000-DMG in a specific molar ratio. The specific formulations are described in the Embodiments. Unless otherwise specified, the lipid components are measured in mmol. Lipids are dissolved in anhydrous ethanol, and the nucleic acids are dissolved in a citrate aqueous solution (10 mM, pH 4.0). The aqueous and organic solutions are mixed at a 3:1 volume ratio using a microfluidic chip with a total flow rate greater than 3 mL/min. The LNP formulations are dialyzed overnight with 1×PBS solution, then transferred to glass vials and stored at 4° C. or −20° C. The final mRNA concentration is: 0.1-0.375 μg/μl. The structure of the prepared LNP^{⊕} formulations is shown in FIG. 16.

### Embodiment 2

### Determination of Encapsulation Efficiency for LNP and LNP^{⊕}

The Triton concentration used in conventional LNP encapsulation efficiency detection methods cannot resolve the LNP^{⊕} formulations.

### 2.1: Effect of Triton X-100 Concentration on RNA/DNA Fluorescence Quantification

Experiment Description: The Qubit HS RNA assay, Qubit 2.0 for RNA content may be affected by Triton X-100. To investigate the impact of different Triton concentrations on the RNA quantification, the present invention first determine the RNA quantification in solutions with varying Triton X-100 concentrations. Specifically, Triton X-100 at different concentrations are mixed with a detection diluent containing 270 ng RNA to prepare test samples with final Triton concentrations of 0.1%, 0.05%, 0.01%, 0.005%, 0.002%, and 0.001% (all are volume percentage concentrations here) These are quantified using Qubit 2.0, and the results are shown in Table 1.

TABLE 1
Effect of Triton X-100 on Qubit HS RNA Kit Detection Results

| Final concentration | | | | | |
|---|---|---|---|---|---|
| of Triton in the | | | | | |
| test sample | Test 1 | Test 2 | Test 3 | Average | Deviation |
| 0.0% | 269 | 266 | 266 | 267.0 | — |
| 0.1% | 272 | 270 | 270 | 270.7 | 1.39% |
| 0.05% | 271 | 270 | 271 | 270.7 | 1.39% |
| 0.01% | 268 | 269 | 267 | 268.0 | 0.37% |
| 0.005% | 274 | 275 | 272 | 273.7 | 2.51% |
| 0.002% | 274 | 274 | 273 | 273.7 | 2.51% |
| 0.001% | 275 | 276 | 272 | 274.3 | 2.73% |

Conclusion: The calibrated RNA concentration is 267.0 ng/ml. Triton X-100 at final concentrations from 0.001% to 0.1% has no significant effect on the RNA quantification results using the Qubit HS RNA Kit, with detection variation deviations less than 3%. Given the dilution factors, the Triton concentrations range from 0% to 20% can be used for RNA detection samples. Similar results are obtained repeatedly and confirmed for plasmid DNA quantification using Qubit HS dsDNA Kit.

2.2: Complete Resolution of Nucleic Acids and Lipid Components in LNP^{⊕} with 10% Triton X-100

### Method for Determining Encapsulation Efficiency of LNP and LNP^{⊕} Formulation

According to the present invention, the encapsulation efficiency of LNP and LNP^{⊕} formulations are determined using Qubit RNA HS Assay Kit (Invitrogen, Q32852) and Qubit dsDNA HS Assay Kit (Invitrogen, Q32851). The RNA or DNA content in the nucleic acid lipid nanoparticles is quantified using the Qubit 2.0 fluorometer. The operation steps are as follows:
a) Measure the nucleic acid content of the lysed sample. Add an equal volume of 20% Triton X-100 solution prepared in 1×TE to the LNP or LNP^{⊕} formulation sample to be tested, mix and centrifuge, and incubate at room temperature in the dark for 5 minutes. Dilute the sample by 200-fold, load the sample for testing, and measure the total nucleic acid concentration (A) in the lysed sample.
b) Without adding Triton detergent, measure the free nucleic acid content in the LNP or LNP^{⊕} sample to obtain the unlysed sample nucleic acid concentration (B).
c) Calculation formula: Encapsulation Efficiency (%)=((A−B)/A)×100.

In the above calculation formula, A: the nucleic acid amount measured in the final concentration at 10% Triton; B: the nucleic acid amount measured in the test solution without Triton.

Typically, LNP formulations composed of ionizable lipids are fully lysed in 1% Triton solution, and the total and free nucleic acid contents are measured and compared using nucleic acid fluorescent dye colorimetry to determine the LNP encapsulation efficiency. After incorporating non-ionized cationic lipids, the stability of the LNP^{⊕} formulation increases, and 1% Triton cannot dissociate the nucleic acids and lipid components in the LNP^{⊕} formulation.

In the LNP encapsulation efficiency detection methods, 2% Triton concentration is the highest concentration reported in the literature so far but still cannot fully dissociate the nucleic acids and lipids in LNP^{⊕} formulations.

The present invention carries out testing for 0%, 1%, 5%, 7.5%, and 10% Triton solutions on LNP^{⊕} formulations with an RNA concentration of 0.1 μg/μL. The nucleic acid content detection results for different formulations of LNP^{⊕} components are shown in Table 2.

TABLE 2
RNA content in RNA-LNP and RNA-LNP^{⊕} samples dissociated by different Triton concentrations

| | | LNP 17 | LNP^{⊕}28 | LNP^{⊕}25 | LNP^{⊕}31 | LNP^{⊕}32 | LNP^{⊕}33 | LNP^{⊕}34 | LNP^{⊕}35 | LNP^{⊕}37 |
|---|---|---|---|---|---|---|---|---|---|---|
| Cationic | Dotap | | 46.29 | 46.29 | 46.29 | 46.29 | 46.29 | 46.29 | 46.29 | 69.44 |
| Lipid | | | | | | | | | | |
| Ionizable | ALC-0315 | 46.29 | 46.29 | 46.29 | 46.29 | 46.29 | 46.29 | 46.29 | 46.29 | 46.29 |
| Lipid | | | | | | | | | | |
| Aux | Cholesterol | 42.67 | 85.34 | 85.34 | 85.34 | 85.34 | 92.6 | 101.85 | 111.1 | 85.34 |
| Lipid | DSPC | 9.4 | 9.4 | — | — | — | — | — | — | 9.4 |
| | PEG-DMG | 1.64 | 3 | 4 | 5 | 5.5 | 4 | 4 | 4 | 4.5 |
| 1% Triton | A Conc. | 0.100 | 0.029 | 0.033 | 0.029 | 0.041 | 0.051 | 0.031 | 0.031 | 0.044 |
| 5% Triton | A Conc. | 0.103 | 0.032 | 0.054 | 0.042 | 0.044 | 0.059 | 0.061 | 0.049 | 0.032 |
| 7.5% Triton | A Conc. | 0.098 | 0.081 | 0.085 | 0.073 | 0.88 | 0.86 | 0.81 | 0.082 | 0.077 |
| 10% Triton | A Conc. | 0.112 | 0.101 | 0.102 | 0.116 | 0.102 | 0.104 | 0.113 | 0.101 | 0.112 |
| 0% Triton | B Conc. | 0.016 | 0.001 | 0.001 | 0.003 | 0.001 | 0.001 | 0.002 | 0.001 | 0.001 |
| EE (%) | ((A − B)/ | 85.451 | 99.335 | 98.965 | 97.446 | 98.58 | 99.067 | 97.879 | 99.278 | 98.635 |
| | A) × 100 | | | | | | | | | |

(EE: Encapsulation Efficiency)

In Table 2, the unit of lipid component concentrations is mmol; the concentration A and the concentration B are the RNA concentrations before and after dissociation in the LNP^{⊕} (or LNP) formulations, in μg/μL.

It is worth mentioning that in Table 2, there is only one B value and one A value available (i.e., the A value measured in 10% Triton is valid for all formulations; 1% Triton is only effective for legacy LNPs containing only ionizable lipids).

Conclusion: 1% Triton solution can fully lyse the LNPs composed of ionizable lipids (ALC-0315) (i.e., LNP17 in Table 2), but cannot dissociate LNP^{⊕} formulations containing cationic lipids (like DOTAP) (i.e., LNP^{⊕} in Table 2, except LNP17). The lysis rate of the LNP^{⊕} formulations tends to increase with higher Triton concentrations. 10% Triton solution fully lyses LNP^{⊕} containing DOTAP, with the measured total nucleic acid content comparable to the total nucleic acid content in the sample. Increasing PEG concentration to 5.5 mmol, cholesterol concentration to 111.1 mmol, or DOTAP concentration to 69.44 mmol does not affect the dissociation ability of 10% Triton solution. 10% Triton solution can be used to completely lyse the nucleic acid and lipid components in LNP^{⊕} without affecting RNA quantification using the Qubit HS RNA Kit.

A comparative experiment using plasmid DNA-LNP^{⊕} yields similar results, as shown in Table 3, where 10% Triton solution does not affect plasmid DNA quantification using the Qubit HS dsDNA Kit.

TABLE 3
DNA content in DNA-LNP and DNA-LNP^{⊕} formulation samples
after dissociation with different Triton concentrations

| | Plasmid | LNP 17 | LNP^{⊕}25 | | |
|---|---|---|---|---|---|
| | DNA (μg) | 15 | 30 | 15 | 7.5 |
| 1% Triton | A Conc. | 0.111 | 0.0044 | 0.0023 | 0.0082 |
| 10% Triton | A Conc. | 0.118 | 0.315 | 0.153 | 0.072 |
| 0% Triton | B Conc. | 0.00073 | 0.00113 | 0.00080 | 0.003 |
| Encapsulation | ((A − B)/ | 99.39 | 99.64 | 99.48 | 99.28 |
| Efficiency (%) | A) × 100 | | | | |

In Table 3, the concentration A and the concentration B are the DNA concentrations before and after dissociation in the LNP^{⊕} (or LNP), in μg/μL.

Additionally, it is observed in the results of this embodiment that the addition of high concentration of non-ionized cationic lipids increases the encapsulation efficiency of the LNP^{⊕} formulation, as seen in Table 2.

### Embodiment 3

### Effects of Ionizable Lipid Concentration on Gene Expression and Distribution Under Intramuscular Injection Administration

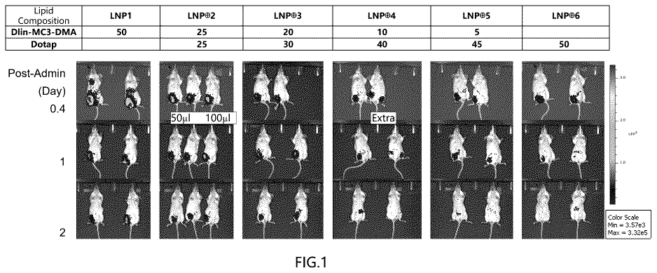

In this embodiment, the lipid particle LNP1 formulation listed in FIG. 1 contains one ionizable lipid MC3, which is one of the formulations used in marketed nucleic acid drug. Without altering the original ratio of the four components (MC3:DSPC:Chol:PEG=50:10:38.5:1.5), this embodiment adjusts the molar ratio of MC3 from 50% to 0%, and compensates with DOTAP, keeping the total concentration of MC3 and DOTAP lipids in the lipid formulation constant. These lipid formulations are used to encapsulate luciferase mRNA, creating a series of LNPs with varying MC3 molar concentrations.

In this embodiment, seven-week-old female Balb/c mice are divided into six groups, with two mice per group, and the drug is administered via intramuscular injection in the right hind limb at a dose of 7.5 μg (mRNA)/50 μl. Six lipid nano preparations, LNP1, LNP^{⊕}2, LNP^{⊕}3, LNP^{⊕}4, LNP^{⊕}5, and LNP^{⊕}6, are tested and the luciferase expression in mice at different time points after intramuscular injection are assessed. In vivo IVIS imaging analysis is performed at 6, 24, 48, and 72 hours post-administration, with imaging results at 6, 24, and 48 hours shown in FIG. 1. In FIG. 1, the molar ratio of LNP components is: (MC3+DOTAP):DSPC:Chol:PEG=50:10:38.5:1.5. Table 4 shows the lipid amounts required for formulations encapsulating 30 μg mRNA, with the lipid measured in mmol.

TABLE 4
Effect of MC3 concentration on tracer gene expression
levels, Unit: fluorescence intensity/p/s

| | LNP1 | LNP^{⊕}2 | LNP^{⊕}3 | LNP^{⊕}4 | LNP^{⊕}5 | LNP^{⊕}6 |
|---|---|---|---|---|---|---|
| Dlin- | 50 | 25 | 20 | 10 | 5 | |
| MC3-DMA | | | | | | |
| Dotap | | 25 | 30 | 40 | 45 | 50 |

BFI

| | | | | | | |
|---|---|---|---|---|---|---|
| 6 Hr | 867,000 | 176,000 | 66,900 | 37,100 | 47,000 | 64,500 |
| 24 Hr | 145,000 | 125,000 | 81,300 | 18,200 | 19,600 | 12,400 |
| 48 Hr | 103,000 | 28,500 | 23,700 | 22,700 | 19,900 | 21,600 |

This experiment reveals that as the molar percentage of MC3 in the LNP formulation decreases, the luciferase protein expression at the intramuscular injection site gradually decreases, showing a clear dose-dependent delivery trend. DOTAP concentration does not compensate for the reduction in luciferase expression induced by decreasing MC3. LNP formulations with MC3 molar percentages below 10% exhibit significantly reduced duration of tracer gene expression at the injection site.

Conclusion: Under intramuscular administration, the ionizable lipid component in LNP has a clear dose relationship with the delivery and sustained expression of the tracer gene at the intramuscular injection site. The intensity and duration of tracer gene expression increase with higher doses of ionizable lipids. Non-ionizable cationic lipids have a much lower ability to deliver the tracer gene at the intramuscular injection site compared to equivalent doses of ionizable lipids and lack the capacity to maintain long-term expression of the tracer gene. Therefore, maintaining sufficient concentrations and doses of ionizable lipid is essential for LNP vaccines and LNP^{⊕} vaccines administered intramuscularly to sustain exogenous gene expression, which directly affects the stimulation and production of specific antibodies.

### Embodiment 4

### Effect of Non-Ionizable Cationic Lipids on LNP^{⊕} Formulations Delivery Mode and Gene Expression Levels Under Intramuscular Injection Administration:

The present invention explores the changes in tracer gene expression and distribution after adding extra non-ionizable cationic lipids to LNP formulations composed of ionizable lipids and then administering the formulations by intramuscular injection.

### 4.1: Non-Ionizable Cationic Lipid DOTAP Alters the Expression Pattern of LNP Lipid Particles

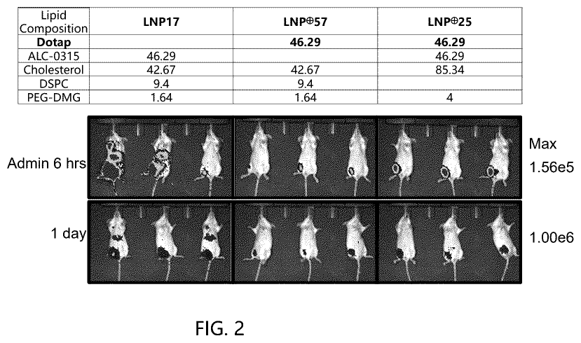

In this embodiment, the lipid particle formulation LNP17 shown in FIG. 2 is one of the marketed mRNA vaccine formulations, containing the ionizable lipid ALC-0315. ALC-0315 in the LNP^{⊕}17 formula is replaced with the non-ionizable cationic lipid DOTAP to form formulation LNP^{⊕}57, which contains only DOTAP lipid. Additionally, DOTAP is added to the LNP^{⊕}17 to create formulations LNP^{⊕}25, LNP^{⊕}46, and LNP^{⊕}74, which are composed of ionizable lipids and non-ionizable cationic lipids.

Seven-week-old female Balb/c mice are divided into seven groups, with three mice per group, and the drug is administered via intramuscular injection in the right hind limb at a dose of 7.5 μg (mRNA)/50 μl. Lipid nanoparticle formulations LNP17, LNP^{⊕}57, and LNP^{⊕}25, etc. are tested separately. In vivo IVIS imaging analysis is performed at 6, 24, 48, 72, and 96 hours post-administration. The results of in vivo IVIS imaging of mice at 6 and 24 hours post-administration are shown in FIG. 2. The formulations in the table of FIG. 2 represent the lipid amounts for encapsulating 30 μg nucleic acid, with the lipids measured in mmol. It should be noted that, unless otherwise specified, the formulations and lipid units in the subsequent figures are the same.

The experimental results show that: under intramuscular administration, the LNP17 group of mice exhibits high level and sustained expression of the tracer gene at the administration site, with the expression signals still detectable 5 days post-administration. At 6 hours post-administration, transient high expression is observed in the visceral tissues of mice, including the liver, chest cavity, and brain tissues, but these expression signals disappear within 24 hours. In the LNP^{⊕}57 group mice, tracer gene expression is significantly reduced, with only weak expression at the intramuscular injection site, and no tracer gene expression is observed after 72 hours. In the LNP^{⊕}25 group of mice, tracer gene expression at the injection site is partially restored, and by 48 hours, the tracer gene expression signal at the intramuscular injection site returns to levels consistent with LNP17. IVIS imaging recorded at 6 hours post-injection intramuscularly shows no tracer gene expression in the abdomen, lungs and major internal organs of mice in the LNP^{⊕}57 or LNP^{⊕}25 formulation groups that contain DOTAP.

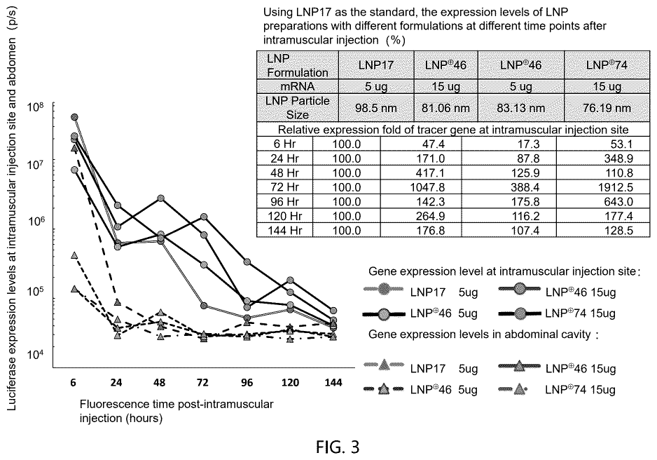

Experimental conclusion: Under intramuscular administration, the sustained high expression of the tracer gene at the administration site and the transient expression in the visceral tissues of mice induced by the LNP formulation depend on the ionizable lipid component. Ionizable cationic lipid ALC-0315 also triggers a strong adjuvant effect. The non-ionizable cationic lipid DOTAP induces a weaker adjuvant effect for inflammation, and the LNP formulations composed of DOTAP show low expression levels in the visceral tissues of mice. The non-ionizable cationic lipid DOTAP can balance the adjuvant effect of the ionizable cationic lipid ALC-0315, reduce the expression levels of the tracer gene in the abdominal cavity of mice by the circulating LNP^{⊕} formulation, and enhance the sustained expression of the tracer gene at the intramuscular injection site to varying degrees, as shown in FIG. 3. The LNP^{⊕} formulations composed of DOTAP are suitable for the nucleic acid vaccine formulations administered via intramuscular injection.

### 4.2: Non-Ionizable Cationic Lipid DOTMA Alters the Expression Pattern of LNP Particles

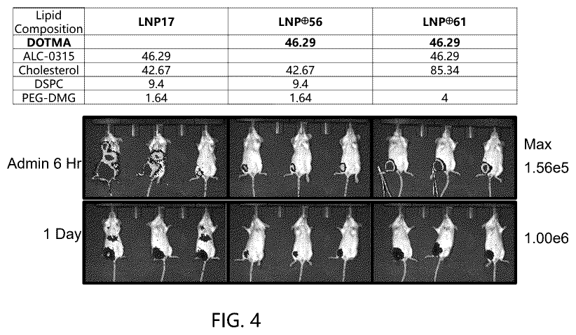

In this embodiment, as shown in FIG. 4, ALC-0315 in the LNP17 formulation is replaced by non-ionizable cationic lipid DOTMA to form formulation LNP^{⊕}56, which contains only DOTMA cationic lipid. Additionally, DOTMA is added to the LNP17 formulation to create formulation LNP^{⊕}61, which consists of ionizable lipids and non-ionized cationic lipids. Using existing microfluidic technology, FLuc mRNA is encapsulated to prepare mRNA-LNP formulations.

Seven-week-old female Balb/c mice are divided into two groups, with 3 mice per group, and the drug is administered by intramuscular injection in the right hind limb at a dose of 7.5 μg (mRNA)/50 μL. Three lipid nanoparticle formulations, including LNP^{⊕}56 and LNP^{⊕}61, are tested separately. In vivo IVIS imaging analysis is performed at 6, 24, 48, and 72 hours post-administration. The results of in vivo IVIS imaging of mice at 6 and 24 hours post-administration are shown in FIG. 4. To reduce the number of animals, this experiment is conducted simultaneously with the DOTAP group, sharing the LNP17 positive control group.

The experimental results show that under intramuscular administration, the LNP^{⊕}56 group exhibit significantly reduced tracer gene expression, with only weak expression at the intramuscular injection site, and the expression level decreased by 622.5 times. No tracer gene expression is observed after 72 hours. In the LNP^{⊕}61 formulation group of mice, tracer gene expression at the injection site is partially restored, and by 24 hours, the tracer gene expression signal at the intramuscular injection site returns to levels consistent with LNP17 and is maintained. IVIS imaging records at 6 hours post-intramuscular injection shows no tracer gene expression in the abdomen, lungs and major internal organs of mice in the LNP^{⊕}56 or LNP^{⊕}61 formulation groups that received DOTMA.

Experimental conclusion: Under intramuscular administration, the adjuvant effect of non-ionizable cationic lipid DOTMA induces a weak adjuvant effect for inflammation, and the LNP^{⊕} formulations composed of DOTMA show low expression levels in the visceral tissues of mice, offering better safety. DOTMA can balance the adjuvant effect of ionizable cationic lipid ALC-0315, reducing the expression level of circulating LNP^{⊕} formulations in the visceral tissues of mice, improving the safety of the formulation, while not affecting the sustained expression ability of the lipid particle formulations at the intramuscular injection site. The LNP^{⊕} formulation composed of DOTMA are suitable for nucleic acid vaccine formulations administered via intramuscular injection.

### 4.3: Universal Expression Pattern of Non-Ionizable Cationic Lipid Particles

In this embodiment, different types of ionizable lipids MC3, DHA-1, L319, and SM-102 are used to replace ALC-0315 in the LNP17 and LNP^{⊕}25 formulations to form various LNPs and LNP^{⊕} formulations. DHA-1 is a branched ionizable cationic lipid provided by SINOPEG (Cat. No.: 06040009300). Furthermore, luciferase mRNA is encapsulated using lipid particles to prepare mRNA-LNP formulation.

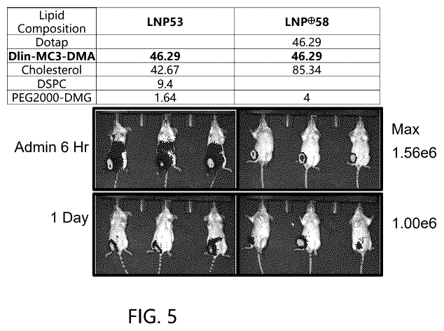

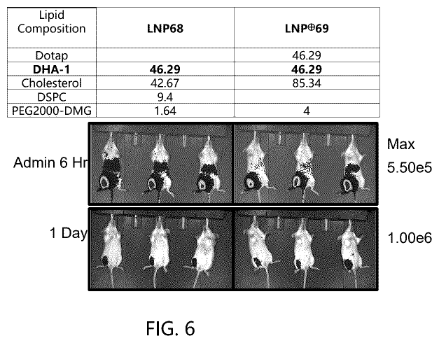

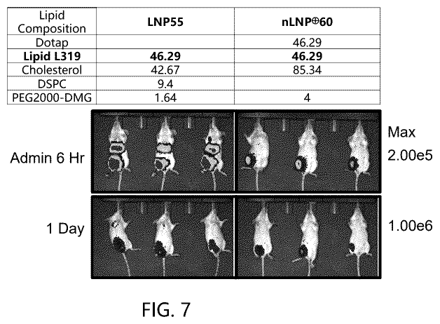

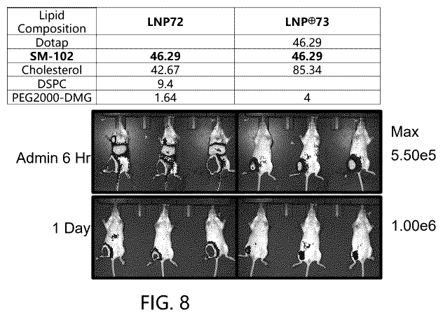

Seven-week-old female Balb/c mice are divided into six groups, with three mice per group, and the drug is administered by intramuscular injection into the right hind limb at a dose of 7.5 μg/50 μl. Six lipid nanoparticle formulations, including LNP53, LNP^{⊕}58, LNP55, LNP^{⊕}60, LNP68, LNP^{⊕}69, LNP72, and LNP^{⊕}73, are tested respectively. In vivo IVIS imaging analysis is performed at 6, 24, 48, and 72 hours post-administration. The results of in vivo IVIS imaging of mice at 6 and 24 hours post-administration are shown in FIGS. 5-8.

The experimental results show that under intramuscular administration, the four ionizable lipid nanoparticle formulations, namely LNP53, LNP55, LNP68 and LNP73, exhibit high and sustained expression of the tracer gene at the administration site, with the expression signal still detectable 3 days post-administration. At 6 hours post-administration, transient high expression is observed in the visceral tissues of mice, including the liver, thoracic cavity, and brain tissues, and the expression signal disappears within 24 hours, accompanied by inflammatory reactions such as redness and swelling. In the groups administered with the four lipid nanoparticle formulations, including LNP^{⊕}58, LNP^{⊕}60, LNP^{⊕}69 and LNP^{⊕}73, which are added with non-ionizable cationic lipids, the tracer gene expression at the injection site of mice is partially restored, and by 48 hours, the tracer gene expression signal at the intramuscular injection site returns to levels consistent with their corresponding positive control groups of mice and is maintained synchronously thereafter. IVIS imaging records at 6 hours post-intramuscular injection show no tracer gene expression in the abdomen, lungs and major internal organs of mice in the groups administered with LNP^{⊕}58, LNP^{⊕}60, LNP^{⊕}69 and LNP^{⊕}73 that received DOTAP.

Experimental conclusion: Under intramuscular injection, incorporating DOTAP into lipid particles composed of ionizable lipids such as MC3, DHA-1, L319, and SM-102 can balance the adjuvant effect of ionizable lipids, reducing systemic off-target expression level, similar to the results observed in the ALC-0315 group. Combined with the similar results from the LNP^{⊕} formulations incorporating DOTMA, by analogy, the results of this experiment can infer that the LNP^{⊕} formulation composed of non-ionizable cationic lipids and ionizable lipids has the ability to reduce the systemic off-target expression level of the target gene in visceral tissues while maintaining sustained expression at the intramuscular injection site.

### Embodiment 5

Effects of cholesterol, phospholipids and PEG components in LNP^{⊕} formulation on gene delivery and expression patterns under intramuscular administration: This embodiment explores the effects of the main components of LNP formulation on tracer gene expression under intramuscular administration.

5.1: Neutral Phospholipids Fine-Tune mRNA-LNP^{⊕} Expression Pattern

In this embodiment, the lipid particle formulations LNP17, LNP^{⊕}25, LNP^{⊕}45, and LNP^{⊕}46 are used to encapsulate FLuc mRNA. The neutral phospholipid DSPC content in the LNP^{⊕} formulations are 0, 9.4, and 18.8 mmol, respectively. The luciferase mRNA is encapsulated to prepare mRNA-LNP formulations.

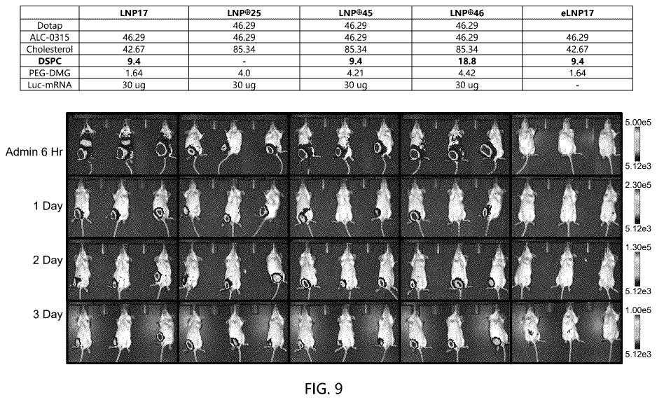

Seven-week-old female Balb/c mice are divided into three groups, with 3 mice per group, and the drug is administered by intramuscular injection in the right hind limb at a dose of 7.5 μg/50 μl. Five lipid nanoparticle formulations, including LNP17, LNP^{⊕}25, LNP^{⊕}45, LNP^{⊕}46, and eLNP17 (empty lipid particles), are tested respectively. In vivo IVIS imaging analysis is performed at 6, 24, 48, 72, 96, and 120 hours post-administration. The results of in vivo IVIS imaging of mice at 6, 24, 48, and 72 hours post-administration are shown in FIG. 9.

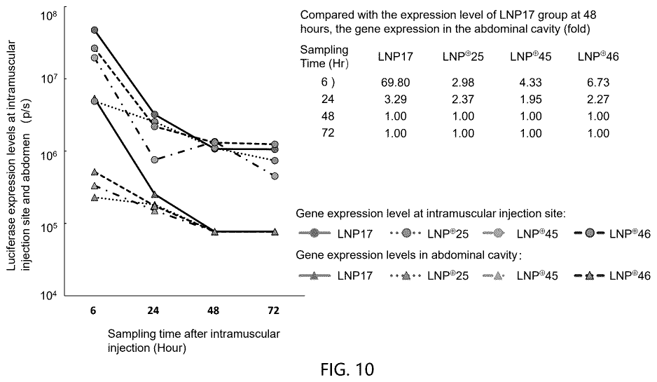

The experimental results show that under intramuscular administration, the LNP17-administered mice exhibit high and sustained expression of the tracer gene at the administration site, with the expression signals still detectable 5 days post-administration. At 6 hours post-administration, transient high expression is observed in the visceral tissues of mice, including the liver, thoracic cavity, and brain tissues, but these expression signals disappeared within 24 hours, accompanied by inflammatory reactions such as redness and swelling. In the group of mice administered with LNP^{⊕}25 with DOTAP, only relatively weak expression is observed at the intramuscular injection site, and no tracer gene expression is detected in the visceral tissues of mice within 24 hours. In the LNP^{⊕}45 and LNP^{⊕}46 groups, expression signals at the injection site and in visceral tissues of mice increase with higher neutral phospholipid concentrations at 6 hours post-administration. However, compared to the abdominal expression signals in LNP17 group mice, the expression levels at the intramuscular injection site decrease by 2.42-fold and 1.78-fold, respectively, while the expression levels in visceral tissues decrease by 16.12-fold and 10.4-fold, respectively. By 48 hours, the tracer gene expression signals at the intramuscular injection site of mice in all LNP^{⊕} formulation administered groups return to the levels consistent with LNP17, and longer observation periods show expression signal levels maintained at the same level or even slightly exceeding those of mice in the LNP17 group, as shown in FIG. 10.

Experimental conclusion: Compared with LNP formulations, intramuscularly administered LNP^{⊕} formulations inhibit the transient expression of the tracer gene in the visceral tissues of mice, but do not affect long-term expression at the intramuscular injection site. This phenomenon is further modulated by the neutral phospholipid component. The decrease in neutral phospholipid concentration causes the LNP^{⊕} formulation to further reduce the transient expression level in the visceral tissues and intramuscular injection site, but do not directly affect the tracer gene expression level at the intramuscular injection site after 24 hours or longer.

### 5.2: Neutral Phospholipids Inhibit Gene Expression in DNA-LNP^{⊕} by

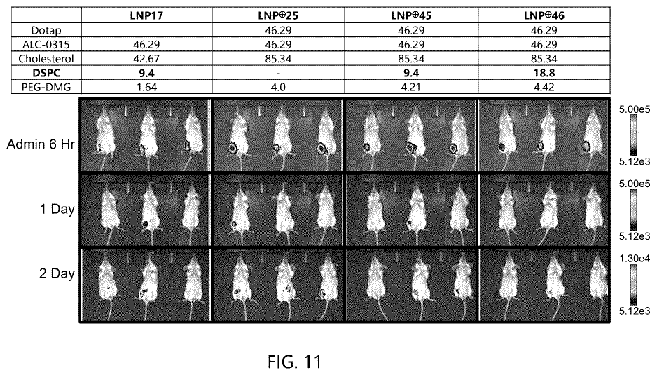

In this embodiment, lipid particle formulations LNP17, LNP^{⊕}25, LNP^{⊕}45, and LNP^{⊕}46 are used to encapsulate FLuc plasmid DNA. As shown in FIG. 11, the neutral phospholipid DSPC contents contained in the DNA-LNP^{⊕} formulations are 9.4, 0, 9.4, and 18.8 mmol, respectively. The luciferase plasmid DNA is encapsulated using the microfluidic chip technology to prepare the DNA-LNP formulations.

Seven-week-old female Balb/c mice are divided into three groups, with three mice per group, and the drug is administered via intramuscular injection in the right hind limb, at a dose of 11.5 μg plasmid DNA/50 μl. Four lipid nanoparticle formulations, DNA-LNP17, DNA-LNP^{⊕}25, DNA-LNP^{⊕}45, and DNA-LNP^{⊕}46, are tested respectively. In vivo IVIS imaging analysis is performed at 6, 24, 48, and 72 hours post-administration. The results of in vivo IVIS imaging of mice at 6, 24, and 48 hours post-administration are shown in FIG. 11.

The experimental results show that at 6 hours post-intramuscular injection, the DNA-LNP17 group exhibit low and poorly sustained expression of tracer gene at the injection site of mice. The mice in the DNA-LNP^{⊕}25 group show the highest expression at the intramuscular injection site, with the expression level at the injection site 3.4-fold increase compared to the mice in the LNP17 group. The mice in the DNA-LNP^{⊕}45 and DNA-LNP^{⊕}46 groups have lower expression signals at the injection site than the mice in the DNA-LNP^{⊕}25 group, decreasing with higher neutral phospholipid concentrations. By 72 hours, the expression signals of the tracer gene are only observed at the intramuscular injection site of the mice in the DNA-LNP^{⊕}25 group. No expression signals of the tracer gene are detected in the visceral tissues in mice of any administration group.

Experimental conclusion: Compared with LNP formulations encapsulating mRNA, the cationic lipid DOTAP enhances the expression level of the tracer gene in DNA-LNP^{⊕} formulations at the intramuscular injection site of mice, while neutral phospholipid components inhibit the expression ability of DNA-LNP^{⊕} formulations. The role of neutral phospholipid components in LNP formulations differs significantly on expression function between mRNA and DNA.

### 5.3: Cholesterol Concentration Range Suitable for Intramuscular Administration of LNP

The present invention compares the effect of cholesterol concentration on the gene delivery ability of LNP^{⊕} formulations. In this embodiment, the mRNA-LNP formulation used has a molar ratio of cationic lipids (including ionizable lipids and non-ionizable cationic lipids) to cholesterol designed to be between 10:7 and 10:12. The luciferase mRNA is encapsulated using microfluidic technology to prepare mRNA-LNP formulations.

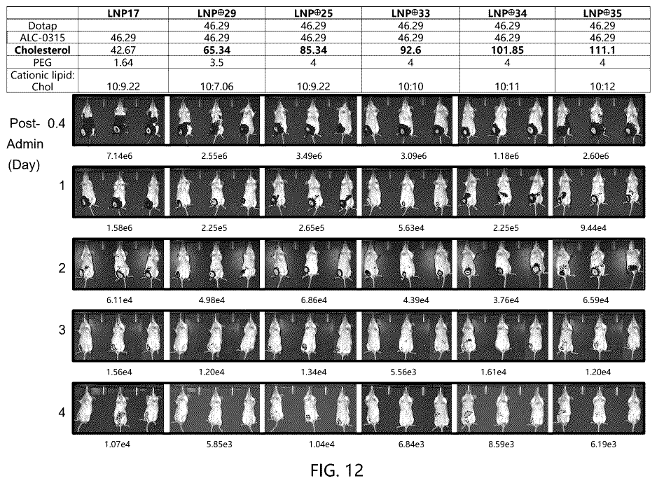

Seven-week-old female Balb/c mice are divided into six groups, with three mice per group, and the drug is administered via intramuscular injection in the right hind limb at a dose of 7.5 μg/50 μl. Six lipid nanoparticle formulations, LNP17, LNP^{⊕}29, LNP^{⊕}25, LNP^{⊕}33, LNP^{⊕}34, and LNP^{⊕}35, are tested respectively. In vivo IVIS imaging analysis is performed at 6, 24, 48, 72, and 96 hours post-administration, and the results are shown in FIG. 12.

The experimental results show that at 6 hours post-intramuscular injection, the change in cholesterol concentration has some effect on the expression level of the LNP^{⊕} formulation compared with the LNP17 group. At 96 hours post-administration, the LNP^{⊕} formulations with a cationic lipid to cholesterol molar ratio designed between 10:9 to 10:11 still exhibit relatively high expression at the intramuscular injection site.

Experimental conclusion: Cholesterol concentration affects the expression persistence of LNP^{⊕} formulations. The molar ratio of cationic lipids (including ionizable lipids and non-ionizable cationic lipids) to cholesterol between 10:9 to 10:11 is favorable for gene expression and maintenance in the LNP^{⊕} formulations.

### 5.4: Effect of PEG Concentration on LNP^{⊕} Expression Patterns

The present invention compares the effect of PEG concentration on the gene delivery ability of LNP^{⊕} formulations. In the mRNA-LNP formulations used in this embodiment, the PEG concentration is designed to be 0.23%, 0.46%, 0.91%, 1.64%, 1.66%, 1.78%, 1.93%, 2.20%, 2.47%, 2.73%, and 3.0% of the total lipid molar amount. The luciferase mRNA is encapsulated by using a microfluidic process to prepare an mRNA-LNP formulation.

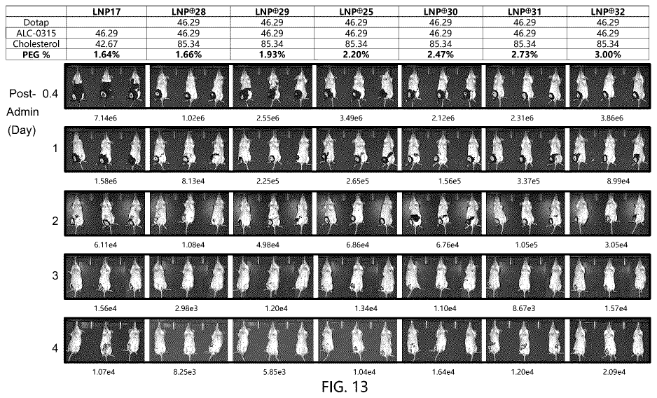

Seven-week-old female Balb/c mice are divided into twelve groups, three mice in each group, and the drug is administered via intramuscular injection in the right hind limb at a dose of 7.5 μg mRNA/50 μl. Twelve lipid nanoparticle formulations, including LNP17, LNP^{⊕}28, LNP^{⊕}25, LNP^{⊕}30, LNP^{⊕}31, and LNP^{⊕}32, are tested respectively. In vivo IVIS imaging analysis is performed at 6, 24, 48, 72, and 96 hours post-administration, with the results shown in FIG. 13. In FIG. 13, the PEG amounts are expressed as the molar percentage of total lipids.

The experimental results show that at 6 hours post-intramuscular injection, all PEG concentrations has no significant effect on the expression level of LNP^{⊕} formulations compared to the LNP17 group. At 72 hours post-administration, LNP^{⊕} formulations with PEG constituting 1.93% to 3.0% of the total lipid molar ratio continue to maintain the expression level, while those with PEG concentrations below 1.93% show a significant decrease in expression levels.

Experimental conclusion: A molar ratio of 2.20% to 3.0% PEG maintains the expression levels of the LNP^{⊕} formulations, which is beneficial for gene expression and maintenance.

### Embodiment 6

Intramuscular Administration of nCovS2P mRNA-LNP Formulations Induces Production of COVID-19 Spike Protein-Specific Antibodies and Neutralizing Antibodies in Balb/C Mice

### 6.1: MRNA-LNP^{⊕} Formulation Stimulate High-Level Immune Responses and Coordinate Humoral Immunity Levels.

According to this embodiment, seven-week-old female BalB/C mice are randomly divided into five groups and vaccinated via intramuscular injection with lipid particle complexes encapsulating mRNA encoding the COVID-19 Spike protein (perfusion conformation locked with S2P mutation). The nCovS2P mRNA coding sequence is identical to that of the Pfizer/BioNTech COVID-19 Spike protein recombinant sequence BNT162b2 mRNA coding sequence. The particle size and encapsulation efficiency of the lipid particles are shown in Table 5. Each group of animals received two injections 3 weeks apart. On day 21 after the first immunization (3wp1), and on days 7, 14, and 21 after the second immunization (1wp2, 2wp2, and 3wp2), mice are anesthetized, blood is collected, and serum samples are tested for IgG antibodies and neutralizing antibodies against the COVID-19 S protein.

TABLE 5
LNP, LNP^{⊕} Particle Size, Particle Size Distribution, and EE % Test results

| | LNP | Nucleic | Particle | | |
|---|---|---|---|---|---|
| Reagent | Formula | Acid | size (nm) | PDI | EE % |
| Fluc@LNP17 | LNP17 | 5 μg FLuc mRNA | 90.83 | 0.1061 | 90.23 |
| nCovS2P@LNP17 | LNP17 | 5 μg nCovS2P mRNA | 74.14 | 0.1028 | 96.61 |
| nCovS2P@LNP^{⊕}46 | LNP^{⊕}46 | 5 μg nCovS2P mRNA | 77.61 | 0.1124 | 96.31 |
| nCovS2P@LNP^{⊕}25 | LNP^{⊕}25 | 5 μg nCovS2P mRNA | 73.79 | 0.1398 | 97.46 |
| nCovS2P@LNP^{⊕}74 | LNP^{⊕}74 | 5 μg nCovS2P mRNA | 81.31 | 0.1162 | 98.71 |

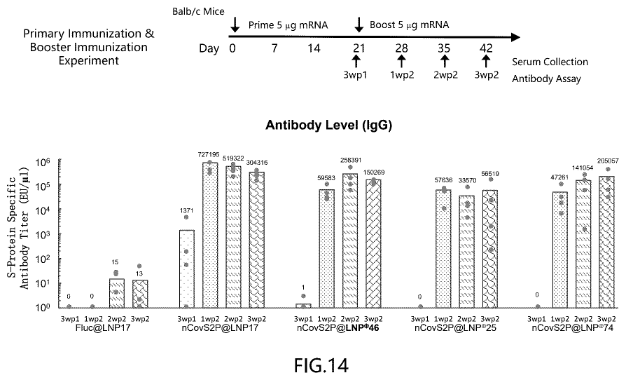

Serum IgG antibody ELISA results show that: 7 days after the second immunization, compared to the blank group, the nCovS2P@LNP17, nCovS2P@LNP^{⊕}46, nCovS2P@LNP^{⊕}25, and nCovS2P@LNP^{⊕}74 administration groups have significant higher levels of serum S protein-specific IgG antibodies in mice serum (p<0.001). The IgG antibody levels in the LNP17 administration group reaches the antibody titer level as reported in the literature and is significantly higher than those in other administration groups (FIG. 14). The serum antibody levels of mice in the LNP^{⊕}46, LNP^{⊕}25, and LNP^{⊕}74 administration groups are 1/20 to 1/100 of those in the LNP17 group, respectively. Notably, compared to the immune effect of traditional vaccines, all LNP^{⊕} formulation groups exhibit extremely high expression levels of S protein-specific antibodies in the serum of mice. 14 days after the second immunization, the specific antibody titer in the serum of mice in the LNP17 group begins to decline, decreasing by approximately fivefold, while the serum antibodies of mice in the LNP^{⊕}46 and LNP^{⊕}74 formulation groups continue to rise, showing a stable increasing trend. 21 days after the second immunization, the S protein-specific IgG antibody levels in the LNP^{⊕}46 and LNP^{⊕}74 groups of mice are significantly closer to those in the LNP17 group, reaching the levels between 50% and 75% of its levels.

### 6.2: MRNA-LNP^{⊕} Formulations Maximize Neutralizing Antibody Levels

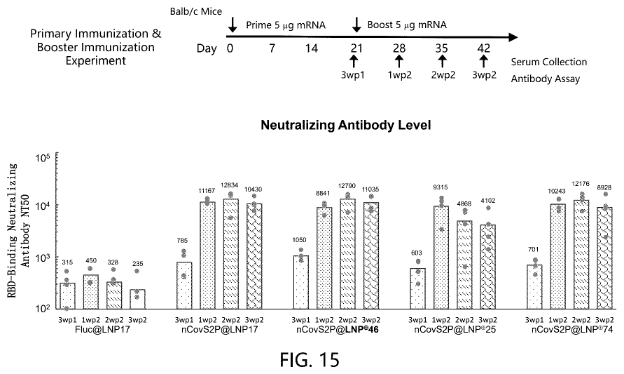

The mouse serum samples obtained from Experiment 6.1 are diluted 1000-fold and tested for RBD competitive neutralizing antibodies using ELISA. The results show that 7 days after the second immunization, compared to the blank group, the LNP17, LNP^{⊕}46, @LNP^{⊕}25, and LNP^{⊕}74 formulation groups have significant higher neutralizing antibody titers in mouse serum (p<0.001). The neutralizing antibody levels in the nCovS2P@LNP17 formulation group are close to the highest peak (FIG. 15). The serum neutralizing antibodies of mice in the LNP^{⊕}46, LNP^{⊕}25, and LNP^{⊕}74 formulation groups are 79.17% to 91.72% of those in the LNP17 group, respectively, indicating extremely high expression levels. 14 and 21 days after the second immunization, the serum neutralizing antibodies of mice in the LNP^{⊕}46 and LNP^{⊕}74 formulation groups show a stable increasing trend, reaching the peak levels of mice in the control group. In the LNP^{⊕}25-formulation group, the level of serum neutralizing antibodies in mice shows a downward trend, with overall level at 40% of the peak.

Combining the analysis of S protein-specific antibodies and RBD-ACE2-binding neutralizing antibodies reveals that, compared to the control LNP formulation, LNP^{⊕}46 and LNP^{⊕}74 formulations induce similar levels of neutralizing antibodies while stimulating lower levels of IgG antibodies. Antigens expressed at the intramuscular injection site have the effect of stimulating neutralizing antibodies. Compared to the LNP^{⊕}46 formulation, the LNP^{⊕}25 formulation, which only expresses at the intramuscular injection site, stimulates lower level of antibodies while maintaining relatively high neutralizing antibody titers. Therefore, by adjusting the formulation of LNP^{⊕} formulations, the dynamics of antigen gene expression at the intramuscular injection site and in visceral tissues can be modulated to regulate the proportion of antibodies (humoral immunity) and neutralizing antibodies produced by the immune system.

### Embodiment 7

Serological Indicators of Acute Toxicological Responses in Balb/C Mice Induced by nCovS2P mRNA-LNP^{⊕} Formulation

According to this embodiment, seven-week-old female BalB/C mice are randomly divided into five groups and administered via intramuscularly injection with nCovS2P-encapsulated lipid particle complexes as formulations. The dose is 20 μg mRNA (or equivalent lipid particles). Grouping information: 1: blank control (1×PBS); 2: eLNP17; 3: nCovS2P@LNP17; 4: nCovS2P@LNP^{⊕}25; 5: nCovS2P@LNP^{⊕}46. Serum is collected at 6 hours, 24 hours, and 48 hours post-administration, and liver-related biochemical indicators are measured in blood samples.

Test results are shown in FIG. 16, and the results analysis is as follows:

1. ALB: The ALB levels of the four groups of mice are consistent with those of the negative control group, with no increase observed.

2. ALT: The ALT levels of mice in the nCovS2P@LNP^{⊕}25, nCovS2P@LNP^{⊕}46 and negative control groups are generally consistent with the control across the three time points, with slight fluctuations but no significant differences. mice in the LNP17 group have significantly higher ALT levels than the control group, especially at 24 and 48 hours, showing a marked increase.

3. TBIL: Overall, the TBIL levels in the three groups of mice, nCovS2P@LNP^{⊕}25, nCovS2P@LNP^{⊕}46 and negative control are slightly higher than the negative control group at 24 and 48 hours, with levels generally consistent among the three groups. However, the LNP17 group show a significant increase in blood TBIL concentration at 24 hours.

4. AST: The AST levels of mice in the nCovS2P@LNP^{⊕}25 group are consistent with the negative control group at three time points, with no significant changes. The mice in the nCovS2P@LNP^{⊕}46 group show a significant increase in AST levels at 24 hours. The mice in the LNP17 group have significantly elevated AST levels at 24 hours, which decrease by 48 hours but the expression is obvious. The blank lipid particle group show significant elevated AST levels at all three time points.

Results analysis: During the experiment, hemolysis and inflammatory reactions such as redness and swelling at the injection site may occur, primarily leading to increased AST levels with impact on other indicators. It cannot be ruled out that the slight increase in AST level is related to the above phenomena. However, combined with the more liver-specific indicators ALT and TBIL, the LNP17 group has higher levels of these two key indicators at each time point compared to the negative control and the LNP^{⊕} formulation group. At 6 hours, there may have been signs of mild liver damage, with significant liver damage observed at 24 and 48 hours, most observed at 24 hours, and showing a downward trend at 48 hours. In contrast, no clear signs of liver damage is observed in the blank control group, the nCovS2P@LNP^{⊕}25 and the nCovS2P@LNP^{⊕}46 formulation groups.

Possible mechanism: Fluorescence experiments indicate that the mRNA-LNP17 formulation show high expression in the liver at 6 hours and 24 hours post-injection, gradually decreasing to negative control group levels around 48 hours. Liver damage may be due to strong immune responses triggered by mRNA expression in the liver, leading to secretion of large amounts of immune factors and overactivation of immune cells, which attack normal liver cells. Therefore, damage continues within 24 hours post-injection, causing liver enzymes to rise continuously. However, after 24 hours, as gene expression in the liver gradually decreases and is limited to the muscle site by 48 hours the immune activation induced attack ceases, allowing the liver to be repaired, and the liver enzymes are gradually metabolized, and improving the indicators. In the control and LNP^{⊕} formulation groups, immune stimulation is primarily at the muscle site with minimal diffusion to the lower abdomen, resulting in little stimulation to the liver and minimal changes in liver enzymes, consistent with the conclusions obtained in this experiment.

What is claimed is:

1. A nucleic acid-lipid nanoparticle suitable for intramuscular administration, characterized in that: it is composed of the following components: (a) at least one nucleic acid; (b) at least one ionizable lipid, constituting 20 mol % to 35 mol % of total lipids; (c) at least one non-ionizable cationic lipid, constituting 15 mol % to 30 mol % of the total lipids; (d) neutral phospholipid or a lipid mixture of its derivatives, constituting 0 mol % to 10 mol % of the total lipids; (e) cholesterol or a mixture of its derivatives, constituting 40 mol % to 56 mol % of the total lipids; (f) PEG or a mixture of PEG and its derivatives, constituting 1.5 mol % to 3 mol % of the total lipids; molecule of (a) the nucleic acid is encapsulated within the lipid nanoparticle composed of (b), (c), (d), (e) and (f).

2. The nucleic acid-lipid nanoparticle suitable for intramuscular administration, according to claim 1, characterized in that: it is composed of the following components: (a) mRNA; (b) one ionizable lipid, constituting 23.01 mol % to 24.17 mol % of the total lipids; (c) at least one non-ionizable cationic lipid, constituting 23.01 mol % to 24.17 mol % of the total lipids; (d) neutral phospholipids, constituting 4.91 mol % to 9.35 mol % of the total lipids; (e) cholesterol, constituting 42.43 mol % to 44.56 mol % of the total lipids; (f) PEG, constituting 2.20 mol % of the total lipids.

3. The nucleic acid-lipid nanoparticle suitable for intramuscular administration, according to claim 1, characterized in that: it is composed of the following components: (a) at least one nucleic acid; (b) at least one ionizable lipid, constituting 20 mol % to 35 mol % of total lipids; (c) at least one non-ionizable cationic lipid, constituting 15 mol % to 30 mol % of the total lipids; (d) cholesterol or a mixture of its derivatives, constituting 40 mol % to 56 mol % of the total lipids; (e) PEG or a mixture of its derivatives, constituting 1.5 mol % to 3 mol % of the total lipids; the molecule of (a) the nucleic acid is encapsulated within the lipid nanoparticle composed of (b), (c), (d) and (e).

4. The nucleic acid-lipid nanoparticle suitable for intramuscular administration, according to claim 3, characterized in that: it is composed of the following components: (a) a mRNA or DNA; (b) an ionizable lipid, constituting 25.45 of the total lipids; (c) a non-ionizable cationic lipid, constituting 25.45 mol % of the total lipids; (d) cholesterol, constituting 46.90 mol % of the total lipids; (e) PEG, constituting 2.20 mol % of the total lipids.

5. The nucleic acid-lipid nanoparticle suitable for intramuscular administration, according to claim 1 or claim 3, characterized in that: the nucleic acid comprises at least an mRNA encoding a polypeptide or an mRNA comprising modified nucleotides.

6. The nucleic acid-lipid nanoparticle suitable for intramuscular administration, according to claim 1 or claim 3, characterized in that: the nucleic acid comprises DNA.

7. The nucleic acid-lipid nanoparticle suitable for intramuscular administration, according to claim 1 or claim 3, characterized in that: the non-ionizable cationic lipid is at least one selected from the group consisting of DOTAP, DOTMA, DC-chol and DOSPA and derivatives thereof.

8. The nucleic acid-lipid nanoparticle suitable for intramuscular administration, according to claim 1 or claim 3, characterized in that: a molar ratio of a sum of the ionizable lipid and the non-ionizable cationic lipid to the cholesterol is from 10:9 to 10:11.

9. The nucleic acid-lipid nanoparticle suitable for intramuscular administration, according to claim 1 or claim 3, characterized in that: a molar concentration of the ionizable lipid is equal to that of the non-ionizable cationic lipid.

10. A formulation prepared from the nucleic acid-lipid nanoparticle according to claim 1 or claim 2, characterized in that: the formulation comprises the nucleic acid-lipid nanoparticle and a pharmaceutically acceptable carrier.

11. The formulation according to claim 10, characterized in that: the formulation is an injection.

12. An application of the nucleic acid-lipid nanoparticle according to claim 1 or claim 2 in the preparation of a biological vaccine.

13. The application according to claim 12, characterized in that: the biological vaccine is a COVID-19 vaccine, an influenza vaccine, or a cancer vaccine.

14. A method for detecting the nucleic acid-lipid nanoparticle according to claim 1 or claim 3, characterized in that: the method comprising: by completely dissolving the nucleic acid-lipid nanoparticles in a solution containing 10 vol % or more Triton, and performing quantitative detection via fluorescence quantification.

15. A reagent, solution and detection kit for use in the method according to claim 14.
